(19) United States
(12) Patent Application Publication
(10) Pub. No.: US 2025/0032004 A1
(43) Pub. Date: Jan. 30, 2025

(54) EXTERNALLY DIRECTED CALIBRATION FOR IMPLANTABLE MEDICAL DEVICE

(71) Applicant: Medtronic, Inc., Minneapolis, MN (US)

(72) Inventors: Jeraz D. Engineer, Minneapolis, MN (US); Jason C. Lee, Edina, MN (US); Michael J. Galloway, Blaine, MN (US); Juliana E. Pronovici, New Hope, MN (US); Ashley L. Garlarneau, Robbinsdale, MN (US); Irah-Vanesa Comia, Denver, CO (US); Mary C. Hallett, Plymouth, MN (US); Ryan D. Wyszynski, Oak Grove, MN (US)

(21) Appl. No.: 18/780,954

(22) Filed: Jul. 23, 2024

Related U.S. Application Data
(60) Provisional application No. 63/516,304, filed on Jul. 28, 2023.

(51) Int. Cl. A61B 5/11; A61B 5/00; A61B 5/283; A61B 5/319; G16H 40/40; G16H 40/67

(52) U.S. Cl. CPC A61B 5/1126; A61B 5/1118; A61B 5/283; A61B 5/319; A61B 5/6801; A61B 5/6846; G16H 40/40; G16H 40/67; A61B 2560/0223; A61B 2562/0219

(57) ABSTRACT

A system includes an implantable medical device and processing circuitry. The implantable medical device includes an accelerometer and sensing circuitry. The processing circuitry obtains, during a calibration period, a first motion signal from the accelerometer. The processing circuitry determines a motion threshold based on the first motion signal. The motion threshold relates to an amount of motion of the patient that is significant for treatment of the health condition of the patient. The processing circuitry obtains, during a collection period, a second motion signal from the accelerometer. Responsive to the second motion signal satisfying the motion threshold, the processing circuitry stores data related to the treatment of the health condition of the patient.

### RELATED APPLICATIONS

This application claims the benefit of U.S. Provisional Application Ser. No. 63/516,304, filed Jul. 28, 2023, the entire contents of each of which are incorporated herein by reference.

### FIELD

The disclosure relates generally to medical systems and, more particularly, medical systems configured to monitor patient health.

### BACKGROUND

Some types of medical systems may monitor various patient data of a patient or a group of patients to detect changes in health. In some examples, the medical system may monitor the data to detect one or more health conditions, such as arrhythmia, heart failure, etc. In some examples, the medical system may include one or more of an implantable medical device or a wearable device to collect the data based on sensing of physiological or other parameters of the patient.

### SUMMARY

The sensors that an implantable medical device or wearable device may use to sense patient parameters may include an accelerometer. Accelerometer data collected by such devices may be used for a variety of purposes, including calibrating the medical device. For example, processing circuitry may use accelerometer data to determine motion thresholds that are each associated with an amount of motion of a patient that is significant for treatment of a health condition. Conventionally, such thresholds are fixed values set by a manufacturer during device development, e.g., based on bench testing and/or pre-clinical studies. Threshold values calibrated for and specific to a patient may be an improvement over such fixed values due to sample size constraints associated with the fixed values, device/sensor variability, and patient/use variability.

Accurate calibration is important for ensuring the performance and safety of medical devices. Improving calibration may correspondingly improve the precision and accuracy of a medical device's measurements. For example, a better-calibrated device can better measure patient parameters. Moreover, accurate calibration may contribute to improved predictability and reliability in device operation. For example, with improved calibration, the medical device's performance may be more consistent, reducing the chances of malfunctions or erratic behavior.

In some examples, a system comprises: an implantable medical device comprising an accelerometer configured to sense motion of a patient; and processing circuitry configured to: obtain a first motion signal generated by the accelerometer during a calibration period, wherein the first motion signal is associated with a directed activity of the patient that relates to an amount of motion of the patient that is significant for treatment of a health condition of the patient; determine a motion threshold based on the first motion signal, wherein the motion threshold relates to the amount of motion of the patient that is significant for treatment of the health condition of the patient; obtain a second motion signal generated by the accelerometer during a collection period; and responsive to the second motion signal satisfying the motion threshold, store data related to the treatment of the health condition of the patient.

In some examples, an implantable medical device comprises: an accelerometer configured to sense motion of a patient; and processing circuitry configured to: obtain a first motion signal generated by the accelerometer during a calibration period, wherein the first motion signal is associated with a directed activity of the patient that relates to an amount of motion of the patient that is significant for treatment of a health condition of the patient; determine a motion threshold based on the first motion signal, wherein the motion threshold relates to the amount of motion of the patient that is significant for treatment of the health condition of the patient; obtain a second motion signal generated by the accelerometer during a collection period; and responsive to the second motion signal satisfying the motion threshold, store data related to the treatment of the health condition of the patient.

In some examples, a method comprises: obtaining, by processing circuitry, a first motion signal generated by an accelerometer of an implantable medical device during a calibration period, wherein the first motion signal is associated with a directed activity of the patient that relates to an amount of motion of the patient that is significant for treatment of a health condition of the patient; determining, by the processing circuitry, a motion threshold based on the first motion signal, wherein the motion threshold relates to the amount of motion of the patient that is significant for treatment of the health condition of the patient; obtaining, by the processing circuitry, a second motion signal generated by the accelerometer during a collection period; and responsive to the second motion signal satisfying the motion threshold, storing, by the processing circuitry, data related to the treatment of the health condition of the patient.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates example environment of an example medical system in conjunction with a patient, in accordance with techniques of this disclosure.

FIG. 2A is a perspective drawing illustrating an insertable cardiac monitor, in accordance with techniques of this disclosure.

FIG. 2B is a perspective drawing illustrating another insertable cardiac monitor, in accordance with techniques of this disclosure.

FIG. 3 is a functional block diagram illustrating an example configuration of a medical device, in accordance with techniques of this disclosure.

FIG. 4 is a functional block diagram illustrating an example configuration of an external device, in accordance with techniques of this disclosure.

FIG. 5 is a block diagram illustrating an example system that includes a network and computing devices, in accordance with techniques of this disclosure.

FIG. 6 is a flow diagram illustrating an example technique for using an example medical system, in accordance with techniques of this disclosure.

Like reference characters denote like elements throughout the description and figures.

### DETAILED DESCRIPTION

In general, medical systems according to this disclosure implement techniques for calibrating thresholds of a medical device that trigger collection of data. An example medical system includes at least one medical device or other sensor device (hereinafter referred to as a medical device) that is configured to collect data using sensors such as motion sensors, electrical sensors, optical sensors, etc. A variety of medical devices (e.g., implantable devices, wearable devices, etc.) may be configured to monitor and store the data for diagnostic purposes.

The medical device may itself implement the techniques of this disclosure to configure the thresholds. In some examples, the medical device may transmit data associated with configuring the thresholds to a computing device or cloud computing system for performing an application of the techniques. Example medical devices in accordance with techniques of this disclosure may include an implantable or wearable monitoring device. Examples of implantable monitoring devices may include the Reveal LINQ™ or LINQ II™ Insertable Cardiac Monitor (ICM), available from Medtronic, Inc. of Minneapolis, MN, a pacemaker/defibrillator, etc.

Some of the techniques described herein may improve the performance of medical systems at classifying health conditions of a patient, such as heart failure. For example, by implementing such improvements, the medical systems and techniques described herein may selectively collect data in a way that increases the accurate detection of health conditions (e.g., by detecting fewer false positives). Furthermore, the techniques described herein may enable the medical device to be calibrated for a particular patient (or patient group). In other words, the techniques described herein may personalize the patient's medical device to detect health conditions more accurately in that patient. Additionally, the techniques described herein may enable the medical device to continuously (e.g., in a periodic and/or event-driven manner) and automatically (e.g., without human intervention) calibrate the sensors, enabling automatic adaptation to changes in patient conditions or patient-device environment that may impact device functionality. This automatic calibration may allow the device to maintain accuracy during the lifetime of the device with little to no clinical intervention.

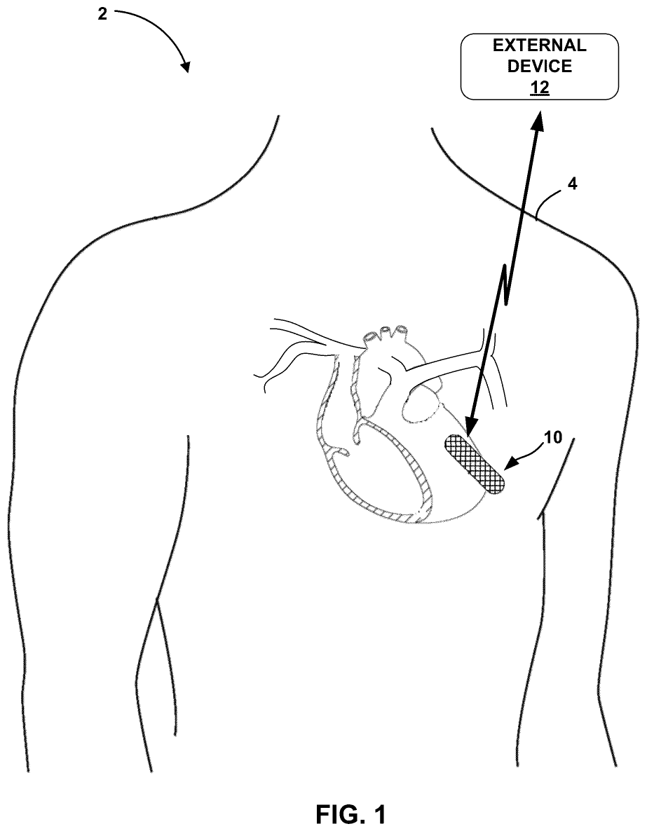

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 may be positioned on other locations, such as patient 4's cranium region. IMD 10 includes one or more sensors (not shown in FIG. 1) and is configured to sense data via the one or more sensors. In some examples, IMD 10 takes the form of the Reveal LINQ™ or LINQ II™ ICM. In some examples, the one or more sensors are configured to sense patient motion/activity, e.g., one or more accelerometers.

External device 12 may be a computing device with a display viewable by the user and an interface for receiving user input to external device 12. In some examples, external device 12 may be a notebook computer, tablet computer, workstation, one or more servers, cellular phone, personal digital assistant, or another computing device that may run an application that enables the computing device to interact with IMD 10. External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

External device 12 may be used to configure operational parameters and/or device settings for IMD 10. External device 12 may be used to retrieve data from IMD 10. The retrieved data may include values of physiological parameters measured by IMD 10, indications of health conditions detected by IMD 10, and physiological signals recorded by IMD 10. As will be discussed in greater detail below, one or more remote computing devices may interact with IMD 10 in a manner similar to external device 12, e.g., to program IMD 10 and/or retrieve data from IMD 10, via a network. External device 12 may be a computing device of patient 4 or a clinician. In some examples, both patient 4 and clinician may interact with respective computing devices to implement the techniques of this disclosure.

In general, sensors of IMD 10 are configured to produce accurate, reliable, and consistent measurements. Accurate measurements are essential to patient safety because IMD 10 may make critical determinations about treatment (e.g., as diagnosing heath conditions, monitoring physiological parameters, and/or delivering medical therapy). However, while the sensors of IMD 10 are calibrated, the sensors are usually calibrated to be suitable for the general population and not a specific patient. Thus, the sensors of IMD 10 may collect less accurate data depending on patient 4. Furthermore, in order to accurately measure data, the sensors may be relatively complex and specialized, which can increase the cost of IMD 10. Additionally, even when calibrated, the sensors may collect less accurate measurements based on noise or artifacts associated with patient activity (e.g., when patient 4 is exercising).

In accordance with techniques of this disclosure, processing circuitry of system 2 may be configured to calibrate thresholds for sensors of IMD 10. External device 12 may instruct IMD 10 to initiate an extrinsic calibration period (“calibration period”). External device 12 may instruct IMD 10 in response to input from patient or another user, such as a clinician, and/or another computing device or system. For example, the clinician may interact with another computing device or cloud computing system, to initiate the calibration period of IMD 10 using external device 12. For example, the clinician may interact with a remote computing system that is not in the same room as the patient. In some examples, the other computing device or computing system may autonomously initiate the calibration period (e.g., in response to a schedule correlated to a date of implantation of IMD 10 in patient, detection of a change in an orientation of IMD 10, etc.). In some examples, system 2 (e.g., IMD 10, external device 12, etc.) may initiate the calibration period in response to detection of a calibration profile (e.g., calibration settings) of IMD 10 deviating from historical data (e.g., existing patient data) by at least a predetermined threshold. In other words, system 2 (e.g., IMD 10, external device 12, etc.) may initiate the calibration period in response to detection of a data from a calibrated sensor or sensors deviating from historical data (e.g., existing patient data) by at least a predetermined threshold.

During the calibration period, external device 12 may instruct patient 4 to perform directed activities (e.g., physical exercises, movements, etc.). Example directed activities may include standing, walking, sitting, reclining, talking, eating, relaxing, running, etc. In some examples, external device 12 may output the instructions for the directed activities via a display of external device 12. Example instructions may include having patient 4 stand for a predetermined amount of time, walk for a predetermined number of steps, etc. Other example instructions may include continuing the calibration protocol until IMD 10 has collected sufficient data to satisfy collection metrics (e.g., metrics that pertain to the quality of data related to the treatment of the health condition of patient 4). For example, IMD 10 may record a motion signal associated with the directed activity until the motion signal (or metrics associated with the motion signal) satisfies collection metrics.

The directed activities may relate to an amount of motion of patient 4 that is significant for treatment of a health condition of patient 4. For example, the amount of motion may be associated with noise in the data collected by IMD 10 (e.g., the signals from sensors of IMD 10). Additionally or alternatively, the amount of motion may indicate that patient 4 is being active, which may relate to a metric that IMD 10 is configured to monitor (e.g., an activity level of patient 4).

In some examples, the directed activities may include or otherwise pertain to key performance indicators calculated based on current parameter value performance. For example, if the calibration profile of IMD 10 is deviating from historical data (e.g., patient 4 transitioning from spending 8 hours per day at a certain time period in supine position to 8 hours per day at an elevated angle at the same time period), IMD 10 may automatically calibrate the sensors to correct the deviation. System 2 may prescribe the directed activities, either based on direct physician input or existing data collected by system 2. For example, system 2 may not prescribe directed activities for a patient with very low activity but prescribe directed activities for a patient with a normal or high activity. In this way, system 2 may facilitate optimizing calibration settings for patients based on patient activity and/or other patient metrics.

IMD 10 may include communication circuitry configured to wirelessly communicate with external device 12 and receive communication from external device 12, e.g., indicating that patient 4 has been instructed to perform a specific directed activity and/or directing IMD 10 to record motion sensor data. The processing circuitry of IMD 10, processing circuitry of external device 12, and/or or other processing circuitry of system 2, may use the information from IMD 10 and external device 12 to associate motion signals with directed activities. For example, the processing circuitry may obtain a first motion signal (e.g., because patient 4 is performing a directed activity). The communication circuitry may receive a communication from external device 12 indicating that patient 4 is performing the directed activity of running. Accordingly, the processing circuitry may associate the first motion signal with the directed activity of running.

The processing circuitry may determine a motion threshold based on a motion signal. For example, the processing circuitry may adjust the motion threshold to attain expected measurements, values, readings, etc., associated with patient 4 performing a directed activity of running. The motion threshold may relate to an amount of motion of patient 4 that is significant for treatment of the health condition of patient 4. For example, the processing circuitry may calibrate the motion threshold based on an amount of motion of patient 4 associated with noise in signals collected by sensors of IMD 10. Additionally or alternatively, the processing circuitry may calibrate the motion threshold based on an amount of motion that indicates patient 4 is being active.

When patient 4 finishes performing the one or more directed activities, the calibration period may end and a collection period may begin. For example, responsive to performing all the directed activities, patient 4 may use external device 12 to instruct IMD 10 to end the calibration period and initiate a collection period. During the collection period, patient 4 may perform daily activities, and IMD 10 may obtain (e.g., from the accelerometer of IMD 10) associated motion signals. For example, during the collection period and while patient 4 is running, IMD 10 may obtain a second motion signal.

IMD 10 may determine whether motion signals obtained during the collection period satisfy the motion threshold and process the motion signals accordingly. For example, responsive to satisfaction of the motion threshold, IMD 10 may store data related to the treatment of a health condition of patient 4, such as patient data. In some examples, IMD 10 may determine that a motion signal, such as the second motion signal, satisfies the motion threshold when the motion signal is equal to or less than the motion threshold. This configuration may be advantageous for reducing noise (e.g., due to motion of patient 4) in the data collected by sensors of IMD 10. In some examples, IMD 10 may determine that a motion signal, such as the second motion signal, satisfies the motion threshold when the motion signal is equal to or greater than the motion threshold. This configuration may be advantageous for monitoring an activity level of patient 4.

As indicated above, satisfaction of the motion threshold may indicate the absence of noise due to excessive motion in signals collected by sensors of IMD 10. Accordingly, responsive to the second motion signal satisfying the motion threshold, the processing circuitry of IMD 10 may store data related to the treatment of the health condition of patient 4. For example, IMD 10 may store signals collected by sensors of IMD 10 relating to patient data, which may include one or more of respiration data, impedance data, posture data, temperature data, blood pressure data, heart rate data, etc. Additionally or alternatively, satisfaction of the motion threshold may indicate that patient 4 is being active (e.g., walking, running, etc.). Accordingly, responsive to the second motion signal satisfying the motion threshold, the processing circuitry of IMD 10 may store activity level data.

In some examples, IMD 10 may determine a periodic activity metric based on a motion signal during a period. In these examples, the motion threshold determined in accordance with the techniques may be a threshold value of the activity metric. For instance, IMD 10 may include a counter to track an activity count as the number of times the signal from an activity sensor crosses the motion threshold during an activity count interval, for example a 2-second interval. Processing circuitry of IMD 10 may correlate the count at the end of each activity count interval to patient body motion during the activity count interval and in turn patient metabolic demand. Methods for obtaining an activity count over an n-second interval and for adjusting the activity sensor signal threshold used for obtaining the activity count are generally disclosed in commonly-assigned U.S. Pat. No. 5,720,769 (van Oort), incorporated herein by reference in its entirety.

Although described in the context of examples in which IMD 10 comprises an ICM, example systems including one or more implantable, wearable, or external devices of any type may be configured to implement the techniques of this disclosure. In some examples, a wearable device operates with IMD 10 and/or external device 12 as potential providers of computing/storage resources and sensors for monitoring patient activity and other patient parameters.

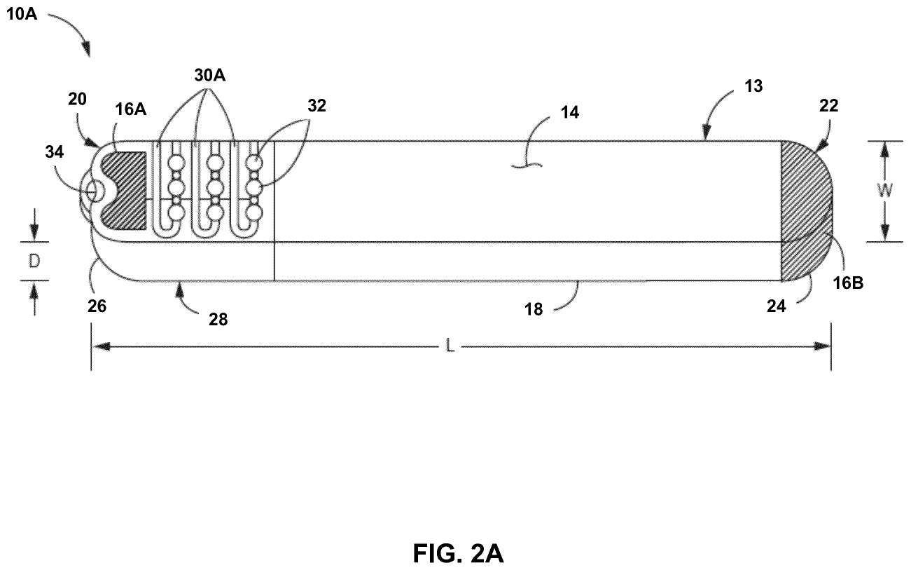

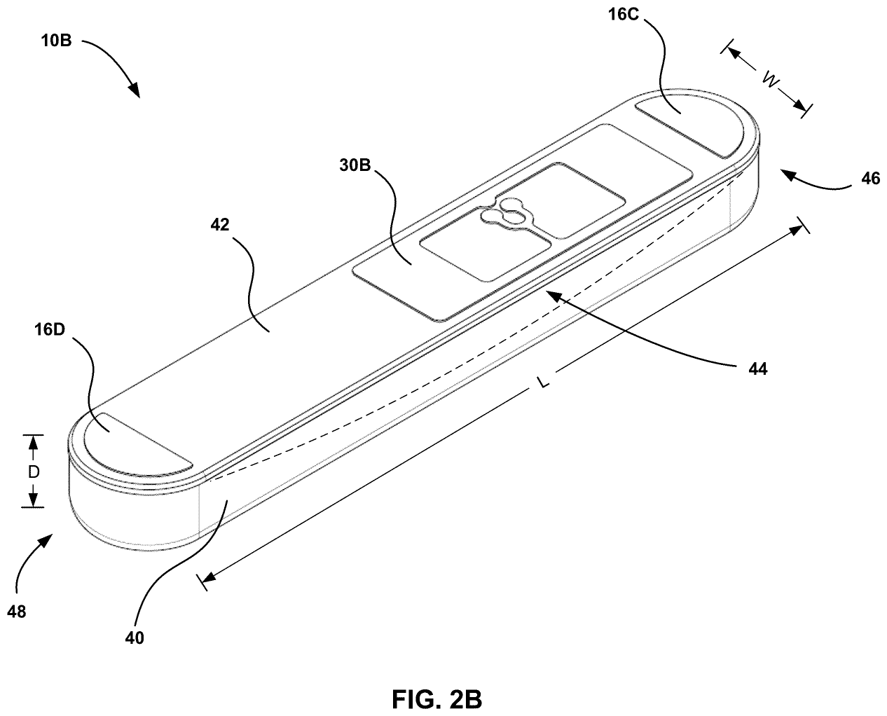

FIG. 2A is a perspective drawing illustrating an IMD 10A, which may be an example configuration of IMD 10 of FIG. 1 as an ICM. In the example shown in FIG. 2A, IMD 10A may be embodied as a monitoring device having housing 13, proximal electrode 16A and distal electrode 16B. Housing 13 may further comprise first major surface 14, second major surface 18, proximal end 20, and distal end 22. Housing 13 encloses electronic circuitry located inside the IMD 10A and protects the circuitry contained therein from body fluids. Housing 13 may be hermetically sealed and configured for subcutaneous implantation. Electrical feedthroughs provide electrical connection of electrodes 16A and 16B.

In the example shown in FIG. 2A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of patient 4 using a minimally invasive procedure and to remain in the desired orientation during insertion. For example, the device shown in FIG. 2A includes radial asymmetries (notably, the rectangular shape) along the longitudinal axis that maintains the device in the proper orientation following insertion. For example, the spacing between proximal electrode 46A and distal electrode 46B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 2A, once inserted within patient 4, the first major surface 14 faces outward, toward the skin of patient 4 while the second major surface 18 is located opposite the first major surface 14. In addition, in the example shown in FIG. 2A, proximal end 20 and distal end 22 are rounded to reduce discomfort and irritation to surrounding tissue once inserted under the skin of patient 4. IMD 10A, including instrument and method for inserting IMD 10 is described, for example, in U.S. Patent Publication No. 2014/0276928, incorporated herein by reference in its entirety.

Proximal electrode 16A is at or proximate to proximal end 20, and distal electrode 16B is at or proximate to distal end 22. Proximal electrode 16A and distal electrode 16B are used to sense cardiac EGM signals, e.g., ECG signals, thoracically outside the ribcage, which may be sub-muscularly or subcutaneously. EGM signals may be stored in a memory of IMD 10A, and data may be transmitted via integrated antenna 30A to another device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, or for measuring impedance, from any implanted location.

In the example shown in FIG. 2A, proximal electrode 16A is at or in close proximity to the proximal end 20 and distal electrode 16B is at or in close proximity to distal end 22. In this example, distal electrode 16B is not limited to a flattened, outward facing surface, but may extend from first major surface 14 around rounded edges 24 and/or end surface 26 and onto the second major surface 18 so that the electrode 16B has a three-dimensional curved configuration. In some examples, electrode 16B is an uninsulated portion of a metallic, e.g., titanium, part of housing 13.

In the example shown in FIG. 2A, proximal electrode 16A is located on first major surface 14 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 14 similar to that shown with respect to proximal electrode 16A.

The various electrode configurations allow for configurations in which proximal electrode 16A and distal electrode 16B are located on both first major surface 14 and second major surface 18. In other configurations, such as that shown in FIG. 2A, only one of proximal electrode 16A and distal electrode 16B is located on both major surfaces 14 and 18, and in still other configurations both proximal electrode 16A and distal electrode 16B are located on one of the first major surface 14 or the second major surface 18 (e.g., proximal electrode 16A located on first major surface 14 while distal electrode 16B is located on second major surface 18). In another example, IMD 10A may include electrodes on both major surface 14 and 18 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 2A, proximal end 20 includes a header assembly 28 that includes one or more of proximal electrode 16A, integrated antenna 30A, anti-migration projections 32, and/or suture hole 34. Integrated antenna 30A is located on the same major surface (i.e., first major surface 14) as proximal electrode 16A and is also included as part of header assembly 28. Integrated antenna 30A allows IMD 10A to transmit and/or receive data. In other examples, integrated antenna 30A may be formed on the opposite major surface as proximal electrode 16A, or may be incorporated within the housing 13 of IMD 10A. In the example shown in FIG. 2A, anti-migration projections 32 are located adjacent to integrated antenna 30A and protrude away from first major surface 14 to prevent longitudinal movement of the device. In the example shown in FIG. 2A, anti-migration projections 32 include a plurality (e.g., nine) small bumps or protrusions extending away from first major surface 14. As discussed above, in other examples anti-migration projections 32 may be located on the opposite major surface as proximal electrode 16A and/or integrated antenna 30A. In addition, in the example shown in FIG. 2A, header assembly 28 includes suture hole 34, which provides another means of securing IMD 10A to patient 4 to prevent movement following insertion. In the example shown, suture hole 34 is located adjacent to proximal electrode 16A. In one example, header assembly 28 is a molded header assembly made from a polymeric or plastic material, which may be integrated or separable from the main portion of IMD 10A.

FIG. 2B is a perspective drawing illustrating another IMD 10B, which may be another example configuration of IMD 10 from FIG. 1 as an ICM. IMD 10B of FIG. 2B may be configured substantially similarly to IMD 10A of FIG. 2A, with differences between them discussed herein.

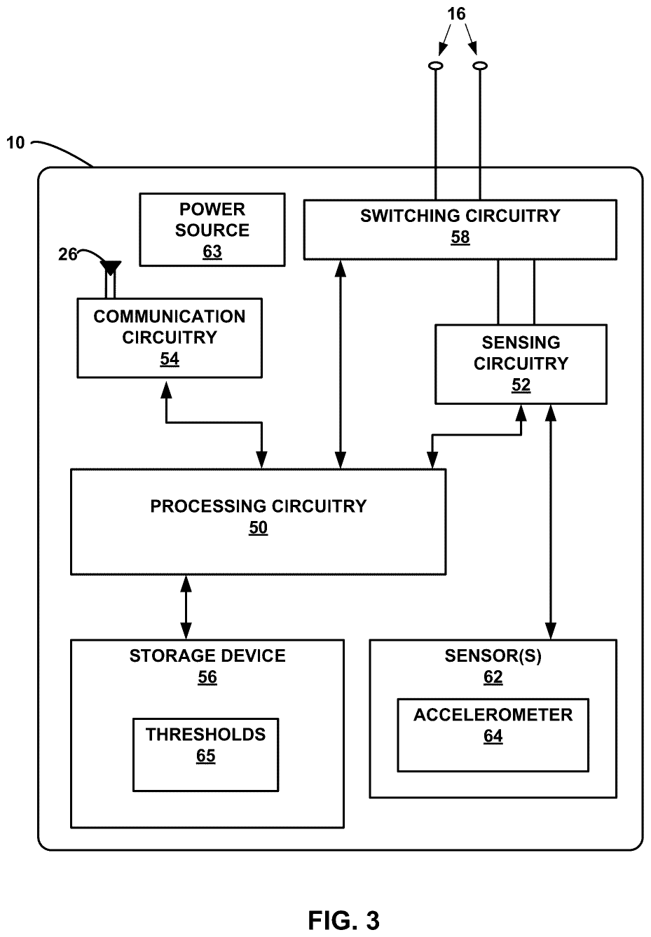

IMD 10B may include a leadless, subcutaneously-implantable monitoring device, e.g. an ICM. IMD 10B includes housing having a base 40 and an insulative cover 42. Proximal electrode 16C and distal electrode 16D may be formed or placed on an outer surface of cover 42. Various circuitries and components of IMD 10B, e.g., described below with respect to FIG. 3, may be formed or placed on an inner surface of cover 42, or within base 40. In some examples, a battery or other power source of IMD 10B may be included within base 40. In the illustrated example, antenna 30B is formed or placed on the outer surface of cover 42, but may be formed or placed on the inner surface in some examples. In some examples, insulative cover 42 may be positioned over an open base 40 such that base 40 and cover 42 enclose the circuitries and other components and protect them from fluids such as body fluids. The housing including base 70 and insulative cover 72 may be hermetically sealed and configured for subcutaneous implantation.

Circuitries and components may be formed on the inner side of insulative cover 42, such as by using flip-chip technology. Insulative cover 42 may be flipped onto a base 40. When flipped and placed onto base 40, the components of IMD 10B formed on the inner side of insulative cover 42 may be positioned in a gap 44 defined by base 40. Electrodes 16C and 16D and antenna 30B may be electrically connected to circuitry formed on the inner side of insulative cover 42 through one or more vias (not shown) formed through insulative cover 42. Insulative cover 42 may be formed of sapphire (i.e., corundum), glass, parylene, and/or any other suitable insulating material. Base 40 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 2B, the housing of IMD 10B defines a length L, a width W and thickness or depth D and is in the form of an elongated rectangular prism wherein the length L is much larger than the width W, which in turn is larger than the depth D, similar to IMD 10A of FIG. 2A. For example, the spacing between proximal electrode 46C and distal electrode 46D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 2B, once inserted subcutaneously within patient 4, outer surface of cover 42 faces outward, toward the skin of patient 4. In addition, as shown in FIG. 2B, proximal end 46 and distal end 48 are rounded to reduce discomfort and irritation to surrounding tissue once inserted under the skin of patient 4. In addition, edges of IMD 10B may be rounded.

FIG. 3 is a functional block diagram illustrating an example configuration of IMD 10 of FIG. 1 in accordance with one or more techniques described herein. In the illustrated example, IMD 10 includes electrodes 16 (e.g., corresponding to any of electrodes 16A-16D), antenna 26, processing circuitry 50, sensing circuitry 52, communication circuitry 54, storage device 56, switching circuitry 58, and sensors 62, which includes one or more accelerometers 64. Processing circuitry 50 may be operatively coupled to sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples. IMD 10 further comprises a power source 64 to provide operational power for processing circuitry 50, sensing circuitry 52, communication circuitry 54, storage device 56, switching circuitry 58, and sensors 62.

Processing circuitry 50, which may be an example of the processing circuitry described in FIG. 1, may be configured to implement functionality and/or execute instructions within IMD 10. For example, processing circuitry 50 may receive and execute instructions that provide the functionality described herein. Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to sense electrical signals of the heart of patient 4, for example by selecting the electrodes 16 and polarity, referred to as the sensing vector, used to sense a cardiac EGM, e.g., ECG, as controlled by processing circuitry 50. Sensing circuitry 52 may sense the cardiac EGM from electrodes 16 in order to facilitate monitoring the electrical activity of the heart. In some examples, sensing circuitry 52 may include one or more filters and amplifiers for filtering and amplifying signals received from electrodes 16 and/or sensors 62. Sensing circuitry 52 and processing circuitry 50 may store patient data in storage device 56, e.g., digitized samples of electrical signals. Sensing circuitry 52 may also monitor signals from sensors 62, which may include one or more accelerometers, pressure sensors, and/or optical sensors, as examples. Sensing circuitry 52 may capture sensor signals from any one of sensors 62, e.g., to produce other patient data, in order to facilitate monitoring of patient activity and detecting changes in patient health.

Communication circuitry 54, which may be an example of the communication circuitry described in FIG. 1, may include any suitable hardware, firmware, software or any combination thereof for wirelessly communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, processing circuitry 50 may control communication circuitry 54 to transmit data (e.g., patient data) to another device, e.g., external device 12 or a cloud computing system comprising one or more computing devices, for analysis. In this manner, the techniques of this disclosure may advantageously enable improved accuracy in the detection of changes in patient health and, consequently, better evaluation of the condition of patient 4.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random-access memory (RAM), read-only memory (ROM), non-volatile RAM (NVRAM), electrically-erasable programmable ROM (EEPROM), ferroelectric RAM (FRAM), dynamic random-access memory (DRAM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include various patient data (e.g., patient physiological parameters such as those described herein). Storage device 56 may store thresholds 65 determined in accordance with techniques of this disclosure.

As described above, processing circuitry 50 may associate motion signals with specific types of directed activities. In some examples, processing circuitry 50 may use these associations to identify the type of activity (“activity type”) patient 4 is performing based on motion signals obtained during the collection period. For example, during the calibration period, communication circuitry 54 may receive a communication from external device 12 that patient 4 is performing a directed activity of walking for 10 seconds. Processing circuitry 50 may calibrate a corresponding first motion threshold based on the motion signal associated with the directed activity of walking for 10 seconds. For example, processing circuitry 50 may adjust the first motion threshold such that the first motion threshold would have been satisfied by the motion signal associated with the directed activity of walking for 10 seconds.

Thus, processing circuitry 50 may count how often patient 4 performs the directed activity of walking by tracking (e.g., a frequency of, a duration of, etc.) satisfaction of the first motion threshold during the collection period. IMD 10 may use a similar process to track other types of directed activities. In this way, processing circuitry 50 may determine an activity count per activity type for patient 4 and in turn determine an activity level of patient 4.

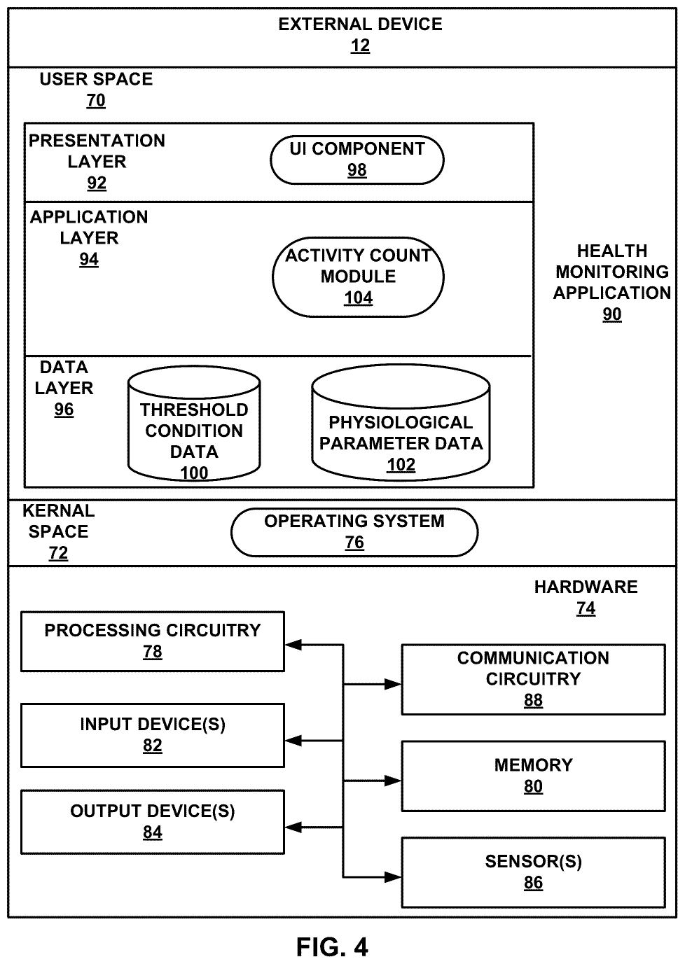

FIG. 4 is a block diagram illustrating an example configuration of external device 12, which, includes a smartphone, a laptop, a tablet computer, a personal digital assistant (PDA), a smartwatch, or any other suitable computing device. As shown in the example of FIG. 4, external device 12 may be logically divided into user space 70, kernel space 72, and hardware 74. Hardware 74 may include one or more hardware components that provide an operating environment for components executing in user space 70 and kernel space 72. User space 70 and kernel space 72 may represent different sections or segmentations of memory, where kernel space 72 provides higher privileges to processes and threads than user space 70. For instance, kernel space 72 may include operating system 76, which operates with higher privileges than components executing in user space 70.

As shown in FIG. 4, hardware 74 includes processing circuitry 78, memory 80, one or more input devices 82, one or more output devices 84, one or more sensors 86, and communication circuitry 88. Although shown in FIG. 4 as a stand-alone device for purposes of example, external device 12 may be any component or system that includes processing circuitry or other suitable computing environment for executing software instructions and, for example, need not necessarily include one or more elements shown in FIG. 4.

Processing circuitry 78 is configured to implement functionality and/or process instructions for execution within external device 12. For example, processing circuitry 78 may be configured to receive and process instructions stored in memory 78 that provide functionality of components included in kernel space 72 and user space 70 to perform one or more operations in accordance with techniques of this disclosure. Examples of processing circuitry 78 may include, any one or more microprocessors, controllers, GPUs, TPUs, DSPs, ASICs, FPGAs, or equivalent discrete or integrated logic circuitry.

Memory 80 may be configured to store information within external device 12, for processing during operation of external device 12. Memory 80, in some examples, is described as a computer-readable storage medium. In some examples, memory 80 includes a temporary memory or a volatile memory. Examples of volatile memories include RAM, DRAM, SRAM, and other forms of volatile memories known in the art. Memory 80, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 80 includes cloud-associated storage.

One or more input devices 82 of external device 12 may receive input, e.g., from a patient, a clinician, or another user. Examples of input are tactile, audio, kinetic, and optical input. Input devices 82 may include, as examples, a mouse, keyboard, voice responsive system, camera, buttons, control pad, microphone, presence-sensitive or touch-sensitive component (e.g., screen), or any other device for detecting input from a user or a machine.

One or more output devices 84 of external device 12 may generate output, e.g., to the patient or another user. Examples of output are tactile, haptic, audio, and visual output. Output devices 84 of external device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 86 may sense physiological parameters or physiological signals of patient 4. Sensor(s) 86 may include electrodes, accelerometers (e.g., 3-axis accelerometers), IMUs, gyroscopes, optical sensors, impedance sensors, temperature sensors, pressure sensors, heart sound sensors (e.g., microphones or accelerometers), and other sensors.

Communication circuitry 88 of external device 12 may communicate with other devices by transmitting and receiving data. Communication circuitry 88 may receive data from IMD 10, such as physiological signals and/or physiological parameter values, from communication circuitry 54 in IMD 10. Communication circuitry 88 may include a network interface card, such as an Ethernet card, an optical transceiver, a radio frequency transceiver, or any other type of device that can send and receive information. For example, communication circuitry 88 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 4, health monitoring application 90 executes in user space 70 of external device 12. Health monitoring application 90 may be logically divided into presentation layer 92, application layer 94, and data layer 96. Presentation layer 92 may include a user interface (UI) component 98, which generates and renders user interfaces of health monitoring application 90.

Data layer 96 may include threshold condition data 100 and physiological parameter data 102, which may be received from IMD 10 via communication circuitry 88 and stored in memory 80 by processing circuitry 78. Threshold condition data 100 may contain threshold conditions (e.g., threshold magnitudes of change, threshold rates of change, threshold periods of time) corresponding to different physiological parameters. External device 12 may receive the thresholds from IMD 10.

External device 12 may determine or receive changes in physiological parameter values and store the changes in physiological parameter values in physiological parameter data 102. In some examples. external device 12 may receive the changes in physiological parameter values from IMD 10. In some examples, external device 12 may determine changes in physiological parameter data 102 by comparing currently sensed physiological parameter values (e.g., by IMD 10) against an average or previously sensed physiological parameter value stored in physiological parameter data 102.

Application layer 94 may include, but is not limited to, an activity count module 104. Activity count module 104 may determine that patient 4 has engaged in an amount of motion significant for health treatment based on satisfaction of one or more threshold conditions stored in threshold condition data 100.

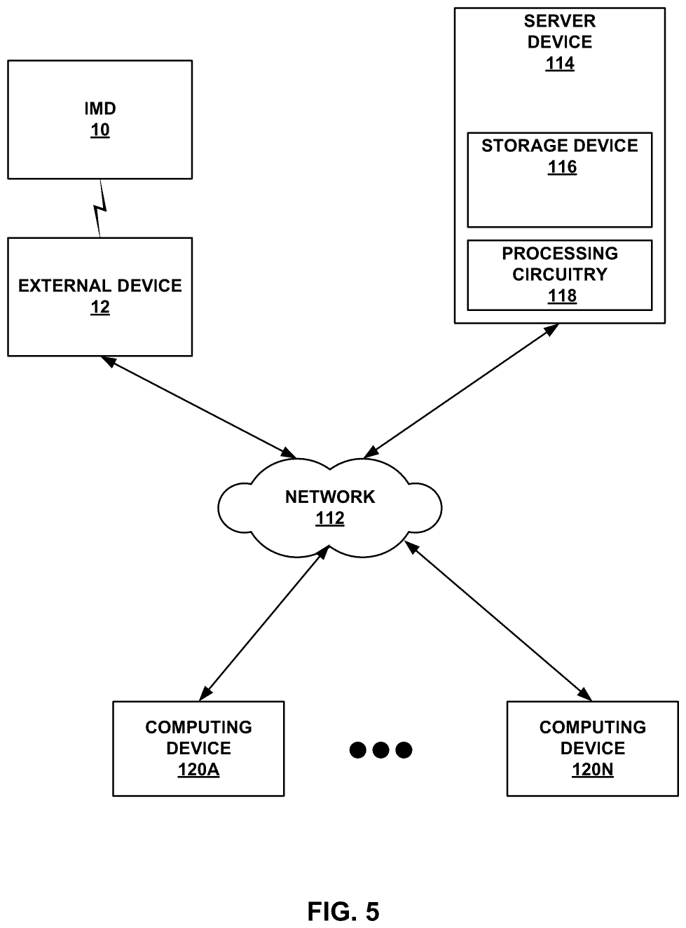

FIG. 5 is a block diagram illustrating an example system that includes external device 12, a network 112, a server 114, and one or more other computing devices 120A-120N (collectively, “computing devices 120”), which may be coupled to IMD 10 and external device 12 via network 112, in accordance with one or more techniques described herein. In this example, IMD 10 may use communication circuitry 54 to communicate with external device 12 via a wireless connection. In the example of FIG. 5, external device 12, server 114, and computing devices 120 are interconnected and may communicate with each other through network 112.

External device 12 may include a device that connects to network 112 via any of a variety of connections, such as telephone dial-up, digital subscriber line (DSL), or cable modem connections. In other examples, external device 12 may be coupled to network 112 through different forms of connections, including wired or wireless connections. In some examples, external device 12 may be a user device, such as a tablet or smartphone, that may be co-located with patient 4. IMD 10 may be configured to transmit data, such as patient data, to external device 12. External device 12 may then communicate the retrieved data to server 114 via network 112.

In some cases, server 114 may be configured to provide a secure storage site for data that has been collected from IMD 10 and/or external device 12. In some cases, server 114 may assemble data in web pages or other documents for viewing by trained professionals, such as clinicians, via computing devices 120. One or more aspects of the illustrated system of FIG. 5 may be implemented with general network technology and functionality, which may be similar to that provided by the Medtronic CareLink® Network.

In some examples, one or more of computing devices 120 may be a tablet or other smart device located with a clinician, by which the clinician may program, receive alerts from, and/or interrogate IMD 10. For example, the clinician may access patient data and/or indications of patient health collected by IMD 10 through a computing device 100, such as when patient 4 is in between clinician visits, to check on a status of a medical condition. In some examples, the clinician may enter instructions for a medical intervention for patient 4 into an application executed by computing device 100, such as based on a status of a patient condition determined by IMD 10, external device 12, server 114, or any combination thereof, or based on other patient data known to the clinician. Device 100 then may transmit the instructions for medical intervention to another of computing devices 120 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 114 includes a storage device 116, e.g., to store data retrieved from IMD 10, and processing circuitry 118. Although not illustrated in FIG. 5 computing devices 120 may similarly include a storage device and processing circuitry. Processing circuitry 118 may include one or more processors that are configured to implement functionality and/or process instructions for execution within server 114. For example, processing circuitry 118 may be capable of processing instructions stored in storage device 116. Processing circuitry 118 may include, for example, microprocessors, DSPs, ASICs, FPGAs, or equivalent discrete or integrated logic circuitry, or a combination of any of the foregoing devices or circuitry. Accordingly, processing circuitry 118 may include any suitable structure, whether in hardware, software, firmware, or any combination thereof, to perform the functions ascribed herein to processing circuitry 118.

Processing circuitry 118 of server 114 and/or the processing circuitry of computing devices 120 may implement any of the techniques described herein to calibrate thresholds of sensors 62 of IMD 10. For example, processing circuitry 118 may receive the motion signals from sensors 62 (e.g., an accelerometer of sensors 62) and determine one or more motion thresholds based on the motion signals. Processing circuitry 118 may receive patient data from IMD 10, external device 12, or other computing device, and processing circuitry 118 may analyze the patient data to treat and monitor a health condition of patient 4.

Storage device 116 may include a computer-readable storage medium or computer-readable storage device. In some examples, storage device 116 includes one or more of a short-term memory or a long-term memory. Storage device 116 may include, for example, RAM, DRAM, SRAM, magnetic discs, optical discs, flash memories, or forms of EPROM or EEPROM. In some examples, storage device 116 is used to store data indicative of instructions for execution by processing circuitry 118.

IMD 10, external device 12, and server 114 may operate together to provide monitoring and feedback. For example, IMD 10 may collect physiological data from the patient's body, such as motion and activity data, as well as motion thresholds. IMD 10 may include sensors and wireless connectivity capabilities, allowing IMD 10 to transmit collected data to external device 12. The frequency and mode of data transmission may vary. In some examples, data transmission may be continuous, at regular intervals, only when specific parameters are satisfied, etc.

External device 12 may serve as an interface for patient 4 and healthcare provider, displaying data from IMD 10 in a user-friendly format. For example, external device 12 may provide real-time feedback, alerts, and alarms to patient 4 and patient data to a clinician. External device 12 may transmit the patient data via network 112 to server 114 for storage and further analysis. Healthcare providers, such as a clinician, may access and evaluate the patient data to determine treatment plans, provide personalized healthcare advice, etc.

Thus, a system in accordance with techniques of this disclosure may include multiple devices that operate together to provide patients with better healthcare. As an example, implementation, IMD 10 may advertise for connection and accept a connection request from external device 12. IMD 10, connecting IMD 10 and external device 12. A clinician using computing device 120A may select a device for interrogating or connecting. In some examples, a clinician may initiate an extrinsic calibration routine using computing device 120A. External device 12 may register the request to initiate the extrinsic calibration routine and send a command to IMD 10. IMD 10 may register the request and in turn initiate a calibration period. External device 12 may receive the confirmation of IMD 10 initiating the calibration period and display directed activities for the patient to perform. In some examples, the clinician may instruct the patient to perform the directed activities.

The patient may perform the directed activities when directed to do so. IMD 10 may acquire the motion data associated with the patient performing the directed activities. IMD 10 may send the motion data (along with timestamps) to external device 12. External device 12 (or another computing device described herein) may compare the data to a range of acceptable values and store the data. External device 12 may then calculate new calibration values and send the calibration data to IMD 10. IMD 10 may accept the new calibration values (e.g., by updating the existing calibration values) and terminate the calibration period.

IMD 10 may inform external device 12 that calibration is complete. External device 12 may mark the calibration routine as complete and, in some examples, inform the clinician (e.g., by sending a notification to computing device 120A). IMD 10 may use the updated calibration values when measuring patient data, which may affect the operation of any of the sensors of IMD 10 as well as determinations based on measurements from the sensors.

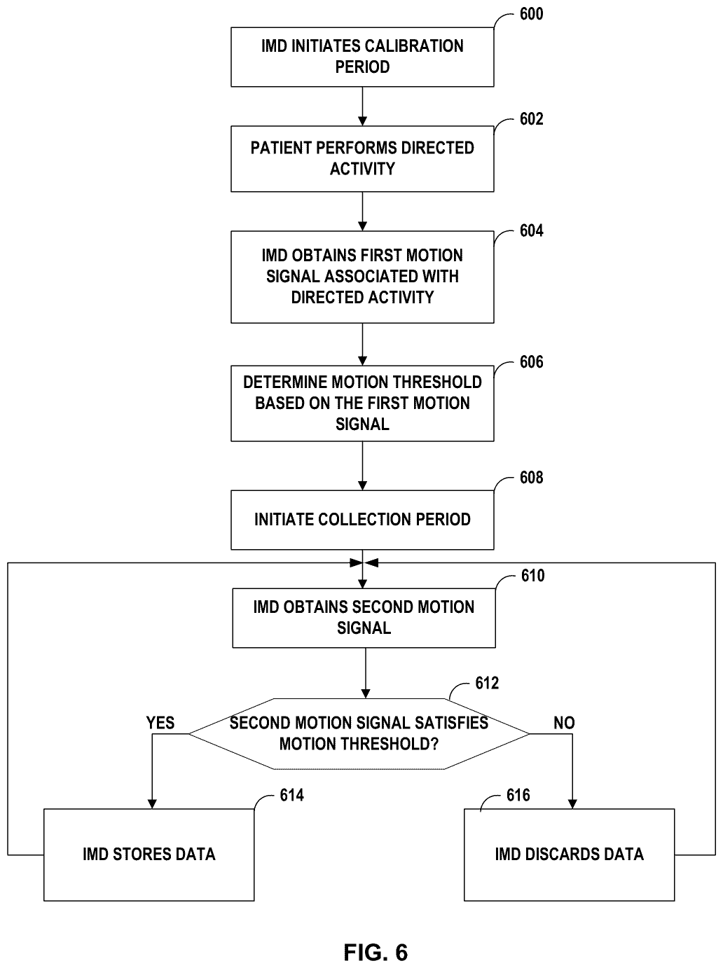

FIG. 6 is a flow diagram illustrating an example technique for using medical system 2. Although primarily described with respect to IMD 10, it should be understood that the techniques of this disclosure may be applied to any medical device described herein.

External device 12 may instruct IMD 10 to initiate a calibration period (600). External device 12 may instruct IMD 10 to initiate the calibration period in response to a user input. During this calibration period, external device 12 may instruct patient 4 to perform directed activities (602). For example, external device 12 may output the instructions for the directed activities via user interface 86 of external device 12. Example instructions may include having patient 4 stand for a predetermined amount of time, walk for a predetermined number of steps, etc.

During the calibration period, processing circuitry 50 may obtain (e.g., from an accelerometer of IMD 10) a first motion signal associated with a directed activity performed by patient 4 (604). At around the same time (e.g., shortly before, during, or shortly after processing circuitry 50 obtains the motion signal), processing circuitry may receive, via communication circuitry 54, a communication from external device 12 indicating that patient 4 is performing a specific directed activity (e.g., a directed activity of walking for 10 seconds). Processing circuitry 50 may use the information from external device 12 to associate the first motion signal with the specific directed activity.

Processing circuitry 50 (or processing circuitry of any other component of the system, such as processing circuitry 78 of external device 12, processing circuitry of server 114, etc.) may determine a motion threshold based on the first motion signal (606). For example, processing circuitry 50 may adjust the motion threshold to attain expected measurements, values, readings, etc., associated with patient 4 performing a directed activity. The motion threshold may relate to an amount of motion of patient 4 that is significant for treatment of the health condition of patient 4. For example, processing circuitry 50 may calibrate the motion threshold based on an amount of motion of patient 4 associated with noise in signals collected by sensors of IMD 10. Additionally or alternatively, processing circuitry 50 may calibrate the motion threshold based on an amount of motion that indicates patient 4 is being active.

When patient 4 finishes performing the one or more directed activities, the calibration period may end, and processing circuitry 50 (Or processing circuitry of any other component of the system, such as processing circuitry 78 of external device 12, processing circuitry of server 114, etc.) may initiate a collection period (608). During the collection period, patient 4 may perform daily activities, and IMD 10 may obtain a second motion signal (610). IMD 10 may determine whether the second motion signal satisfies the motion threshold (612). Responsive to the second motion signal satisfying the motion threshold (“YES” branch of 612), processing circuitry 50 may store data related to the treatment of a health condition of patient 4 (614). The data may include respiration data, impedance data, posture data, temperature data, blood pressure data, heart rate data, etc. Responsive to the second motion signal not satisfying the motion threshold (“NO” branch of 612), processing circuitry 50 may discard the data (616).

Although described as a second motion signal, it should be understood that there is not necessarily a single instance of second signals and that, as depicted in FIG. 6, functions of blocks 612 to 616 might occur repeatedly. For example, IMD 10 may be calibrated in a clinic once and use the calibration results for months or years afterward. In another example, IMD 10 may automatically collect accelerometer signals and update motion detection thresholds periodically without the patient having to follow a specific calibration procedure. For example, processing circuitry 50 may be configured to periodically initiate the calibration period and obtain an updated first motion signal. The first motion signal may be associated with an activity (e.g., a directed activity, an undirected activity, etc.) of the patient that relates to an amount of motion of the patient that is significant for treatment of a health condition of the patient. Processing circuitry 50 may then update the motion threshold based on the updated first motion signal.

The following numbered examples may illustrate one or more aspects of the disclosure:

Example 1: A system includes an implantable medical device includes obtain a first motion signal generated by the accelerometer during a calibration period, wherein the first motion signal is associated with a directed activity of the patient that relates to an amount of motion of the patient that is significant for treatment of a health condition of the patient; determine a motion threshold based on the first motion signal, wherein the motion threshold relates to the amount of motion of the patient that is significant for treatment of the health condition of the patient; obtain a second motion signal generated by the accelerometer during a collection period; and responsive to the second motion signal satisfying the motion threshold, store data related to the treatment of the health condition of the patient.

Example 2: The system of example 1, further includes sensing circuitry configured to collect patient data of a patient, wherein the data related to the treatment of the health condition is the patient data.

Example 3: The system of example 2, wherein the patient data includes at least one of respiration data, impedance data, activity level data, posture data, temperature data, blood pressure data, and heart rate data.

Example 4: The system of any of examples 1 to 3, wherein the second motion signal satisfies the motion threshold when the second motion signal is equal to or less than the motion threshold.

Example 5: The system of any of examples 1 to 3, wherein the second motion signal satisfies the motion threshold when the second motion signal is equal to or greater than the motion threshold.

Example 6: The system of any of examples 1 to 5, further includes wirelessly communicate with an external device; and receive a communication from the external device indicating that the patient is performing the directed activity.

Example 7: The system of any of examples 1 to 6, wherein the motion threshold relates to one or more of noise in the data related to the treatment of the health condition of the patient or that the patient is being active.

Example 8: The system of any of examples 1 to 7, wherein the processing circuitry is further configured to determine an activity count for the directed activity based on one or more of a frequency or duration of satisfaction of the motion threshold.

Example 9: The system of any of examples 1 to 8, wherein the implantable medical device further includes a plurality of sensors, wherein the plurality of sensors includes the accelerometer, and wherein at least one sensor of the plurality of sensors measures the data related to the treatment of the health condition of the patient.

Example 10: The system of any of examples 1 to 9, wherein the implantable medical device includes the processing circuitry.

Example 11: The system of any of examples 1 to 10, wherein an external device includes the processing circuitry.

Example 12: The system of any of examples 1 to 11, wherein a server includes the processing circuitry.

Example 13: The system of any of examples 1 to 12, wherein the implantable medical device includes an insertable cardiac monitor, and wherein the plurality of sensors includes one or more electrodes.

Example 14: The system of example 13, wherein the insertable cardiac monitor includes: a housing configured for subcutaneous implantation in the patient, the housing having a length between 40 millimeters (mm) and 60 mm between a first end and a second end, a width less than the length, and a depth less than the width, wherein the one or more electrodes includes: a first electrode at or proximate to the first end of the housing, and a second electrode at or proximate to the second end of the housing.

Example 15: An implantable medical device includes: an accelerometer configured to sense motion of a patient; and processing circuitry configured to: obtain a first motion signal generated by the accelerometer during a calibration period, wherein the first motion signal is associated with a directed activity of the patient that relates to an amount of motion of the patient that is significant for treatment of a health condition of the patient; determine a motion threshold based on the first motion signal, wherein the motion threshold relates to the amount of motion of the patient that is significant for treatment of the health condition of the patient; obtain a second motion signal generated by the accelerometer during a collection period; and responsive to the second motion signal satisfying the motion threshold, store data related to the treatment of the health condition of the patient.

Example 16: The implantable medical device of example 15, further includes sensing circuitry configured to collect patient data of a patient, wherein the data related to the treatment of the health condition is the patient data.

Example 17: The implantable medical device of example 16, wherein the patient data includes at least one of respiration data, impedance data, activity level data, posture data, temperature data, blood pressure data, and heart rate data.

Example 18: The implantable medical device of any of examples 15 to 17, wherein the second motion signal satisfies the motion threshold when the second motion signal is equal to or less than the motion threshold.

Example 19: The implantable medical device of any of examples 15 to 18, wherein the second motion signal satisfies the motion threshold when the second motion signal is equal to or greater than the motion threshold.

Example 20: The implantable medical device of any of examples 15 to 19, further includes wirelessly communicate with an external device; and receive a communication from the external device indicating that the patient is performing the directed activity.

Example 21: The implantable medical device of any of examples 15 to 20, wherein the motion threshold relates to one or more of noise in the data related to the treatment of the health condition of the patient or that the patient is being active.

Example 22: The implantable medical device of any of examples 15 to 21, wherein the processing circuitry is further configured to determine an activity count for the directed activity based on one or more of a frequency or duration of satisfaction of the motion threshold.

Example 23: The implantable medical device of any of examples 15 to 22, wherein the implantable medical device further includes a plurality of sensors, wherein the plurality of sensors includes the accelerometer, and wherein at least one sensor of the plurality of sensors measures the data related to the treatment of the health condition of the patient.

Example 24: The implantable medical device of any of examples 15 to 23, wherein the implantable medical device includes an insertable cardiac monitor, and wherein the plurality of sensors includes one or more electrodes.

Example 25: The implantable medical device of example 24, wherein the insertable cardiac monitor includes: a housing configured for subcutaneous implantation in the patient, the housing having a length between 40 millimeters (mm) and 60 mm between a first end and a second end, a width less than the length, and a depth less than the width, wherein the one or more electrodes includes: a first electrode at or proximate to the first end of the housing, and a second electrode at or proximate to the second end of the housing.

Example 26: A method includes obtaining, by processing circuitry, a first motion signal generated by an accelerometer of an implantable medical device during a calibration period, wherein the first motion signal is associated with a directed activity of the patient that relates to an amount of motion of the patient that is significant for treatment of a health condition of the patient; determining, by the processing circuitry, a motion threshold based on the first motion signal, wherein the motion threshold relates to the amount of motion of the patient that is significant for treatment of the health condition of the patient; obtaining, by the processing circuitry, a second motion signal generated by the accelerometer during a collection period; and responsive to the second motion signal satisfying the motion threshold, storing, by the processing circuitry, data related to the treatment of the health condition of the patient.

Example 27: The method of example 26, further includes collecting, by sensing circuitry, patient data of a patient, wherein the data related to the treatment of the health condition is the patient data.

Example 28: The method of example 27, wherein the patient data includes at least one of respiration data, impedance data, activity level data, posture data, temperature data, blood pressure data, and heart rate data.

Example 29: The method of any of example 26 to 28, wherein the second motion signal satisfies the motion threshold when the second motion signal is equal to or less than the motion threshold.

Example 30: The method of any of example 26 to 29, wherein the second motion signal satisfies the motion threshold when the second motion signal is equal to or greater than the motion threshold.

Example 31: The method of any of example 26 to 30, further includes wirelessly communicating, by communication circuitry, with an external device; and receiving, by the communication circuitry, a communication from the external device indicating that the patient is performing the directed activity.

Example 32: The method of any of example 26 to 31, wherein the motion threshold relates to one or more of noise in the data related to the treatment of the health condition of the patient or that the patient is being active.

Example 33: The method of any of example 26 to 32, further including determining, by the processing circuitry, an activity count for the directed activity based on one or more of a frequency or duration of satisfaction of the motion threshold.

Example 34: The method of any of example 26 to 33, wherein the implantable medical device further includes a plurality of sensors, wherein the plurality of sensors includes the accelerometer, and wherein at least one sensor of the plurality of sensors measures the data related to the treatment of the health condition of the patient.

Example 35: The method of any of example 26 to 34, wherein the implantable medical device includes the processing circuitry.

Example 36: The method of any of example 26 to 35, wherein an external device includes the processing circuitry.

Example 37: The method of any of example 26 to 36, wherein a server includes the processing circuitry.

Example 38: The method of any of example 26 to 37, wherein the implantable medical device includes an insertable cardiac monitor, and wherein the plurality of sensors includes one or more electrodes.

Example 39: The method of example 38, wherein the insertable cardiac monitor includes: a housing configured for subcutaneous implantation in the patient, the housing having a length between 40 millimeters (mm) and 60 mm between a first end and a second end, a width less than the length, and a depth less than the width, wherein the one or more electrodes includes: a first electrode at or proximate to the first end of the housing, and a second electrode at or proximate to the second end of the housing.

Example 40: A system includes a medical device includes obtain a first motion signal generated by the accelerometer during a calibration period, wherein the first motion signal is associated with a directed activity of the patient that relates to an amount of motion of the patient that is significant for treatment of a health condition of the patient; determine a motion threshold based on the first motion signal, wherein the motion threshold relates to the amount of motion of the patient that is significant for treatment of the health condition of the patient; obtain a second motion signal generated by the accelerometer during a collection period; and responsive to the second motion signal satisfying the motion threshold, store data related to the treatment of the health condition of the patient.

Example 41: The system of example 40, wherein the medical device is a wearable device.

Example 42: The system of example 40 or 41, wherein the processing circuitry is configured to initiate the calibration period in response to detection of a change in an orientation of the medical device.

Example 43: The system of any of examples 40 to 42, wherein the processing circuitry is configured to initiate the calibration period in response to detection of a calibration profile of the medical device deviating from historical data by at least a predetermined threshold.

Example 44: The system of any of examples 40 to 43, wherein the directed activity is based on clinician input or historical data collected by the medical device.

Example 45: The system of any of examples 40 to 44, wherein the processing circuitry is configured to obtain the first motion signal by recording the first motion signal until the first motion signal satisfies one or more collection metrics.

Example 46: The system of any of examples 40 to 45, wherein the processing circuitry is configured to periodically initiate the calibration period; obtain an updated first motion signal, wherein the first motion signal is associated with an activity of the patient that relates to the amount of motion of the patient that is significant for treatment of the health condition of the patient; and update the motion threshold based on the updated first motion signal.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. A system comprising:
a medical device comprising an accelerometer configured to sense motion of a patient; and
processing circuitry configured to:
obtain a first motion signal generated by the accelerometer during a calibration period, wherein the first motion signal is associated with a directed activity of the patient that relates to an amount of motion of the patient that is significant for treatment of a health condition of the patient;
determine a motion threshold based on the first motion signal, wherein the motion threshold relates to the amount of motion of the patient that is significant for treatment of the health condition of the patient;
obtain a second motion signal generated by the accelerometer during a collection period; and
responsive to the second motion signal satisfying the motion threshold, store data related to the treatment of the health condition of the patient.

2. The system of claim 1, further comprising:
sensing circuitry configured to collect patient data of a patient, wherein the data related to the treatment of the health condition is the patient data.

3. The system of claim 2, wherein the patient data comprises at least one of respiration data, impedance data, activity level data, posture data, temperature data, blood pressure data, and heart rate data.

4. The system of claim 1, wherein the second motion signal satisfies the motion threshold when the second motion signal is equal to or less than the motion threshold.

5. The system of claim 1, wherein the second motion signal satisfies the motion threshold when the second motion signal is equal to or greater than the motion threshold.

6. The system of claim 1, further comprising communication circuitry configured to:
wirelessly communicate with an external device; and
receive a communication from the external device indicating that the patient is performing the directed activity.

7. The system of claim 1, wherein the motion threshold relates to one or more of noise in the data related to the treatment of the health condition of the patient or that the patient is being active.

8. The system of claim 1, wherein the processing circuitry is further configured to determine an activity count for the directed activity based on one or more of a frequency or duration of satisfaction of the motion threshold.

9. The system of claim 1, wherein the medical device further comprises a plurality of sensors, wherein the plurality of sensors comprises the accelerometer, and wherein at least one sensor of the plurality of sensors measures the data related to the treatment of the health condition of the patient.

10. The system of claim 1, wherein the medical device is an implantable device.

11. The system of claim 1, wherein a medical device is a wearable device.

12. The system of claim 1, wherein the processing circuitry is configured to initiate the calibration period in response to detection of a change in an orientation of the medical device.

13. The system of claim 1, wherein the processing circuitry is configured to initiate the calibration period in response to detection of a calibration profile of the medical device deviating from historical data by at least a predetermined threshold.

14. The system of claim 1, wherein the directed activity is based on clinician input or historical data collected by the medical device.

15. The system of claim 1, wherein the processing circuitry is configured to obtain the first motion signal by recording the first motion signal until the first motion signal satisfies one or more collection metrics.

16. The system of claim 1, wherein the processing circuitry is configured to:
periodically initiate the calibration period;
obtain an updated first motion signal, wherein the first motion signal is associated with an activity of the patient that relates to the amount of motion of the patient that is significant for treatment of the health condition of the patient; and
update the motion threshold based on the updated first motion signal.

17. An implantable medical device comprises:
an accelerometer configured to sense motion of a patient; and
processing circuitry configured to:
obtain a first motion signal generated by the accelerometer during a calibration period, wherein the first motion signal is associated with a directed activity of the patient that relates to an amount of motion of the patient that is significant for treatment of a health condition of the patient;
determine a motion threshold based on the first motion signal, wherein the motion threshold relates to the amount of motion of the patient that is significant for treatment of the health condition of the patient;
obtain a second motion signal generated by the accelerometer during a collection period; and
responsive to the second motion signal satisfying the motion threshold, store data related to the treatment of the health condition of the patient.

18. The implantable medical device of claim 17, wherein the implantable medical device comprises an insertable cardiac monitor, and wherein the plurality of sensors comprises one or more electrodes.

19. The implantable medical device of claim 18, wherein the insertable cardiac monitor comprises:
a housing configured for subcutaneous implantation in the patient, the housing having a length between 40 millimeters (mm) and 60 mm between a first end and a second end, a width less than the length, and a depth less than the width,
wherein the one or more electrodes comprises:
a first electrode at or proximate to the first end of the housing, and
a second electrode at or proximate to the second end of the housing.

20. A method comprising:
obtaining, by processing circuitry, a first motion signal generated by an accelerometer of an implantable medical device during a calibration period, wherein the first motion signal is associated with a directed activity of the patient that relates to an amount of motion of the patient that is significant for treatment of a health condition of the patient;
determining, by the processing circuitry, a motion threshold based on the first motion signal, wherein the motion threshold relates to the amount of motion of the patient that is significant for treatment of the health condition of the patient;
obtaining, by the processing circuitry, a second motion signal generated by the accelerometer during a collection period; and
responsive to the second motion signal satisfying the motion threshold, storing, by the processing circuitry, data related to the treatment of the health condition of the patient.
